(19) United States
(12) Patent Application Publication
(10) Pub. No.: US 2014/0018610 A1
(43) Pub. Date: Jan. 16, 2014

(54) ENDOSCOPIC MESH DELIVERY SYSTEM WITH INTEGRAL MESH STABILIZER AND VAGINAL PROBE

(71) Applicants: Walter von Pechmann, Bethesda, MD (US); Samuel C. Yoon, Clarksville, MD (US); Keith Lipford, Baltimore, MD (US); Brian Lipford, Bel Air, MD (US); Ausin Cox, Baltimore, MD (US)

(72) Inventors: Walter von Pechmann, Bethesda, MD (US); Samuel C. Yoon, Clarksville, MD (US); Keith Lipford, Baltimore, MD (US); Brian Lipford, Bel Air, MD (US); Ausin Cox, Baltimore, MD (US)

(21) Appl. No.: 13/863,491

(22) Filed: Apr. 16, 2013

Related U.S. Application Data
(63) Continuation-in-part of application No. 12/973,189, filed on Dec. 20, 2010.
(60) Provisional application No. 61/638,256, filed on Apr. 25, 2012.

(51) Int. Cl. A61F 2/00; A61B 17/00

(52) U.S. Cl. CPC A61F 2/0063; A61B 17/00234
USPC 600/37

(57) ABSTRACT

A mesh delivery system for sacral colpopexy and other procedures involving surgical mesh is disclosed. The system uses a mesh stabilizer (30) that is introduced in a compressed configuration through a surgical port into the abdomen, and a vaginal probe (10) (inserted through the vagina) with a magnetic or non-magnetic head that engages with the mesh stabilizer (30), anchoring it in position. The mesh stabilizer (30) employs a pseudoelastic shape memory alloy, and folds compact to deliver multiple mesh straps or a single Y-shaped surgical mesh in a streamlined configuration into the abdomen for facilitating the sacral colpopexy procedure. After delivery, the stabilizer (30) expands to a functional configuration where it interfaces with the probe (10) head and stabilizes and adjustably feeds the mesh strap(s) in preparation for fixation to the vaginal muscularis while maintaining stabilization of the mesh on the vaginal muscularis and while keeping excess mesh from obscuring the surgeons view. After fixation of the mesh to the vaginal muscularis, the stabilizer can be removed back through the surgical port.

### CROSS-REFERENCE TO RELATED APPLICATIONS

The present application derives priority from U.S. Provisional Patent Application 61/638,256 filed 25 Apr. 2012, and is a continuation-in-part of U.S. application Ser. No. 12/973,189 filed 20 Dec. 2010.

### BACKGROUND OF THE INVENTION

1. Field of the Invention

The present invention relates to medical methods and devices for performing sacral colpopexy.

2. Background Art

The sacral colpopexy operation is designed to recreate support to the upper vagina by attaching straps of permanent synthetic mesh to the upper anterior and posterior vaginal walls and then suspending the other end of the straps on the anterior surface of the sacrum. This operation is one of many operations described for the correction of pelvic organ prolapse but is considered the gold standard for correction of prolapse of the upper vagina. See, for example, “Long-Term Success of Abdominal Sacral Colpopexy Using Synthetic Mesh”, Culligan et al. Am J Obstet Gynecol (December 2002). This operation can be done either for correction of vaginal vault prolapse in patients who have previously undergone hysterectomy or can be done at the time of hysterectomy in patients with uterine prolapse. In the latter case, many physicians prefer to perform supracervical hysterectomy because of data suggesting that mesh related complications are less likely in cases of supracervical compared with total hysterectomy.

The sacral colpopexy operation was first described as being done through a large incision in the abdominal wall (laparotomy) and is still predominantly done in that manner.

FIG. 1 is a diagrammatic illustration of the surgery, which is usually performed under general anesthesia. An incision is made in the lower abdomen. The bladder and rectum are freed from the vagina and permanent mesh is secured to the sacrum (upper tailbone) to support the front and back wall of the vagina. The mesh is sutured to the vagina. The peritoneum (lining of the abdominal cavity) is closed over the mesh. There is growing interest in performing this operation via less invasive approaches, such as laparoscopy or robot-assisted laparoscopic surgery, but existing vaginal probes, surgical instruments and mesh configurations are not well-suited for this.

There are a variety of vaginal probes designed for use in treating disorders of the female pelvic floor such as pelvic organ prolapse, urinary incontinence, and sexual dysfunction.

For example, U.S. Pat. No. 6,741,895 to Gafni et al. (Medoc Ltd.) issued May 25, 2004 shows a vaginal probe and method for stimulation of the nerves of the vagina with the purpose of testing their reaction to stimuli in the hope of defining, and treating sexual dysfunction in women. A balloon structure is used to provide tactile stimuli. When the balloon is inflated, these projections poke into the vagina.

United States Patent Application 20060199994 by Inman et al. (AMS Research) issued Sep. 7, 2006 shows surgical instruments useful in pelvic floor repair procedures. The claims require a handle attached to a slender, metal, curved rod.

United States Patent Application 20030220538 to Jacquetin issued 27 Nov. 2003 discloses a particular mesh implant for treating anterior vaginal prolapse.

U.S. Pat. No. 6,932,759 to Kammerer et al. issued Aug. 23, 2005 shows a surgical instrument and method for treating female urinary incontinence with a curved needle-like element and a proximal tape, or mesh, for implanting into the lower abdomen of a female to provide support to the urethra. A second curved needle element is used for simultaneous attachment to the distal end of the first needle.

The IVS Tunneller™ device is available from U.S. Surgical of Norwalk, Conn. The IVS device comprises a fixed delta wing handle, a hollow metal tube and a stylet that is placeable within the tube. The stylet has a rounded plastic tip on one end and an eyelet at the other end. The device may be used to implant a polypropylene tape for infracoccygeal sacropexy and other surgical procedures.

Although the foregoing references have some relevance, they are not suitable for sacral colpopexy, and would not be useful in this latter context. U.S. Pat. No. 6,328,729 (General Surgical Innovations) to Jervis issued Dec. 11, 2001 shows a colporrhaphy method and apparatus in which a tunneling member is advanced and a balloon inflated, thereby dissecting the anatomical space. Again, this device is designed to facilitate dissection of anatomical spaces and is not useful for sacral colpopexy.

Sacral colpopexy has been performed laparoscopically through multiple ports, in one case three to four ports for a daVinci® robot, and one or two ports for the assistant. The polypropylene mesh was attached robotically to the sacral promontory and to the vaginal apex using Gortex™ sutures. Whether performed manually or robotically, there are still inherent problems with manipulating the end effectors and stabilizing the vagina.

Synthetic mesh is commonly used in treatment of pelvic organ prolapse to create a “hammock” to lift the prolapsed organ and return it to its normal position. Similar synthetic or biological meshes are also used in hernia repair to cover the hernia defect, as well as in repair of abdominal wall defects, and abdominal reconstruction. In prolapse repair polypropylene knitted mesh fabrics are most common, and these are woven from monofilament yarns. There are a variety of prolapse repair meshes on the market such as, Popmesh™ by Caldera, Timesh™ by PFM, Avaulta by Bard, Polyform™ by Boston Scientific, Gynecare Gynemesh by Ethicon, and IntePro® Lite. The IntePro® Lite, one of the most commonly used meshes, is made of knitted monofilament polypropylene. It is manufactured by American Medical Systems. In hernia repairs many different synthetic or biologic materials have been proposed over time with varying pore sizes and monofilament fiber compositions, but no single material has gained universal acceptance. In the prolapse context, the available mesh configurations are typically elongate strips, or pre-formed U-shaped or Y-shaped patches with two arms for placement on the sacrospinous ligaments (in the latter case the main stem is placed over the rectovaginal fascia and perineal body).

For example, United States Patent Application 20060015001 to Staskin et al. (American Medical) issued Jan. 19, 2006 shows a sling delivery system to treat urological disorders. The U-shaped configuration of the sling assembly also allows the sling to be adjusted during and/or after implantation. This device is designed for treatment of incontinence and neither it nor any of the foregoing devices are suitable for performance of sacral colpopexy.

United States Patent Application 20030195386 to Thierfelder et al. (AMS Research Corporation) issued Oct. 16, 2003 shows a surgical kit useful for performing a surgical procedure such as a sacral colpopexy with an implantable Y-shaped suspension for treating pelvic floor disorders such as vaginal vault prolapse. AMS also has a device called the Straight-In™ System which uses a long slender instrument designed for endoscopic use that screws a small coil of wire through the pre-formed Y-graft mesh and into the sacrum, thereby obviating the need to suture the mesh to the anterior longitudinal ligament of the sacrum.

FIG. 2 is a front view of an exemplary single-strip mesh 3 and Y-strip 4.

In all configurations the mesh is either sutured or stapled in place. Regardless of mesh composition, pore size or configuration, it is always crucial to lay the mesh over the site without tension and to make sure that it does not fold or bunch up in the process. The mesh being sutured too tight or bunching are common causes of complications. This requires a combination of a reliable mesh dispensing system and stabilizer for stabilizing the mesh in the desired position during suturing or otherwise permanently affixing (e.g.—surgical adhesive) of the mesh to the vagina.

Performing the operation laparoscopically using currently available equipment has several inefficiencies. One of the problematic areas in performing laparoscopic or robotic sacral colpopexy is introduction and positioning of the mesh straps during permanent fixation of the mesh to the vagina. Introduction and dispensing of the mesh straps into the body cavity is difficult using laparoscopic instruments and bunching easily occurs. Guiding them into proper orientation is equally awkward. Maintaining them in the proper position during suturing or otherwise permanently affixing (e.g.—surgical adhesive) requires constant vigilance on the part of the assistant as they frequently require repositioning. Additionally, maintaining the mesh straps in position occupies one or more instruments that could be utilized elsewhere (for instance in retracting the surrounding tissues for better visualization). Sometimes portions of the mesh will drape over and obscure the site of interest, particularly during fixation of the posterior strap of mesh to the posterior vaginal wall.

It has been proposed in other contexts to stabilize one surgical instrument using a second instrument inserted through another incision. For example, U.S. Pat. No. 7,052,453 to Presthus et al. (Solorant Medical) issued May 30, 2006 shows an incontinence treatment with urethral guide that docks with a probe. Generally, the guide can be inserted into a first body orifice and the probe can be inserted into a second body orifice and placed in a predetermined position relative to the guide so as to position the treatment surface adjacent the target tissue in the second body orifice. The urethral guide and probe may align RF sensors relative to a tissue surface.

It would be greatly advantageous to provide a mesh delivery system that overcomes the alignment and positioning problems using a docking concept as above, rendering the mesh attachment for sacral colpopexy less complex and potentially, less time consuming. If the operation can be rendered less time consuming, and with a lower learning curve, there is potential for the operation to be transformed in to one that is done primarily laparoscopically (less invasively), similar to what has already occurred with cholecystectomy (removal of the gall bladder) and therefore reduce patient morbidity and hospitalized care.

It would also be advantageous to provide a mesh delivery system for sacral colpopexy and other procedures requiring fixation of a composite, polyester or polypropylene mesh (performed via laparotomy or laparoscopically) including a method of dispensing said mesh from a stabilizer or other device that improves dispensing efficiency, accuracy and reduces clutter, and an apparatus capable of dispensing said mesh in accordance with the method.

### SUMMARY OF THE INVENTION

It is an object of the present invention to provide a mesh delivery system for sacral colpopexy that facilitates attachment of supporting (anterior and posterior) mesh straps.

It is another object to provide a mesh delivery system for sacral colpopexy (performed via laparotomy or laparoscopically) that uses a conventional laparoscopic surgical tool (e.g.—laparoscopic grasper) for introducing the mesh in combination with a mesh stabilizer into docked attachment to a vaginal probe which is placed in the vagina exteriorly, to thereby stabilize the inserted mesh for permanent fixation to the vaginal tissue.

It is another object to provide a mesh stabilizer with onboard supply of surgical mesh capable of minimally invasive laparoscopic or robot-assisted laparoscopic introduction into the abdominal cavity.

It is another object to provide a pseudo-elastic mesh stabilizer formed with shape memory alloy and carrying an onboard supply of surgical mesh for compressed-keyhole introduction into the abdominal cavity, and detachment and expansion to a functional state in which it facilitates dispensation of the mesh as well as suturing or otherwise permanently affixing (e.g.—stapling or surgical adhesive) of the mesh to the anterior and posterior vaginal walls.

It is still another object to provide a pseudo-elastic mesh stabilizer that when surgically inserted into the abdominal body cavity conforms to a vaginal probe inserted into the vagina, docks magnetically to the probe atop the vaginal apex thereby sandwiching the vaginal apex between itself and the probe, and which independently carries the onboard supply of surgical mesh anchoring the mesh in position on the vaginal apex even after release and removal of the inserter, to facilitate repositioning, dispensation and suturing of the mesh to the anterior and posterior vaginal walls.

It is another object to stabilize the vagina in a fixed but adjustable position during dissection of the tissue planes necessary to allow safe attachment of mesh to the vagina without causing injury to the rectum or bladder.

It is another object to stabilize the vagina in a fixed but adjustable position during fixation of mesh to the vagina.

It is another object to stabilize the loose end(s) of the surgical mesh (the end(s) not being sutured to the vaginal tissue) to prevent the loose ends from obscuring the surgeons vision during the procedure.

It is another object to allow the surgical mesh to be adjustably positioned with respect to the mesh stabilizer and the vaginal tissue following placement of the mesh stabilizer on the vaginal apex while maintaining stabilization of the mesh by the mesh stabilizer.

Other objects, features, and advantages of the present invention will become more apparent from the following detailed description of the preferred embodiments and certain modifications thereof in which a mesh delivery system is provided for sacral colpopexy. The system generally comprises an elastic mesh stabilizer having a plurality of deployable arms and a magnetic docking member(s), a packaging cylinder for introduction of the mesh stabilizer through a conventional port into the abodomen using a conventional grasper or introducer, and a vaginal probe with a magnetic probe tip that interfaces with the magnetic docking member(s) of the mesh stabilizer. The probe may be handheld, robotically-held, or adjustably anchored via a supporting framework to a support surface such as the operating table. The vaginally placed probe essentially acts as a stabilizer for the vaginal tissue during dissection of the bladder and rectum away from the vagina and then during suturing or otherwise permanently affixing (e.g.—stapling or surgical adhesive) of mesh to the vagina. When the probe is inserted into the vagina to the vaginal apex, the magnetic portion of the mesh stabilizer is attracted to the magnetic tip of the vaginal probe thereby anchoring the mesh stabilizer to the tissue of the vaginal apex inside the abdominal body cavity. The mesh stabilizer is designed to deliver anterior and posterior mesh strap(s) for sacral colpopexy through a standard laparoscopic port, and then stabilize the mesh straps on the vaginal apex during fixation. It is equally beneficial to use the mesh stabilizer via a laparotomy approach. In general use the mesh stabilizer with onboard supply of mesh is, maintained in a compressed configuration while introduced by a standard introducer/grasper through a laparoscopic port into the abdomen. The mesh stabilizer expands to a functional configuration conforming to the interior of the vaginal apex, and magnetically docks to to the probe therebeneath. The expansion of the mesh stabilizer deploys and unfurls the onboard mesh from a compressed configuration (unwrinkes the mesh) and positions/anchors the mesh interiorly over the vaginal apex. The docking engagement of the mesh stabilizer to probe through the vaginal tissue locks the mesh stabilizer with mesh straps in place in the desired site with the muscular walls of the vagina sandwiched between the vaginal probe and the mesh stabilizer. The endoscopic introducer/grasper is removed, and the independently-anchored mesh stabilizer facilitates suturing or otherwise permanently affixing (e.g.—stapling or surgical adhesive) of the mesh to the anterior and posterior vaginal walls. After permanent fixation of the mesh, the introducer is reinserted into the abdomen and used to retrieve the mesh stabilizer component. The system greatly facilitates suturing or otherwise permanently affixing (e.g.—stapling or surgical adhesive) of the surgical mesh to the vaginal walls and results in a safer, more effective, less invasive procedure.

### BRIEF DESCRIPTION OF DRAWINGS

Other objects, features, and advantages of the present invention will become more apparent from the following detailed description of the preferred embodiments and certain modifications thereof when taken together with the accompanying drawings in which:

FIG. 1 is a diagrammatic illustration of a completed sacral colpopexy surgery in which straps of mesh attached to the upper vagina inferiorly are suspended on the anterior longitudinal ligament of the sacrum superiorly.

FIG. 2 is a front view of an exemplary single-strip mesh 3 and Y-strip 4.

FIG. 3 is a side perspective view of a mesh delivery system (with mesh 3 on the stabilizer 30) according to the invention.

FIG. 4 is a front view of the mesh stabilizer 30 while in a flattened (pre-shaped) configuration (without the mesh 3).

FIG. 5 is a top perspective view of the mesh stabilizer assembly 30 (including mesh 3) with embedded magnetic members (130, 131) encased in a plastic shell 128.

FIG. 6 is a side perspective view of the mesh stabilizer assembly 30 in a compact folded configuration being loaded into the packaging cylinder 137.

FIG. 7 is a side perspective view of the mesh stabilizer assembly 30 inside cartridge 137 being loaded into a surgical port 60.

FIG. 8 illustrates a packing funnel 70 which may be provided to simplify just-in-time packing of the mesh stabilizer 30, into the cartridge 137.

FIG. 9 shows the use of a loading tether 80.

FIG. 10 shows the use of a protective skirt 85

FIG. 11 is a top perspective view of the vaginal probe 10.

FIG. 12 is a side perspective view of the vaginal probe 10.

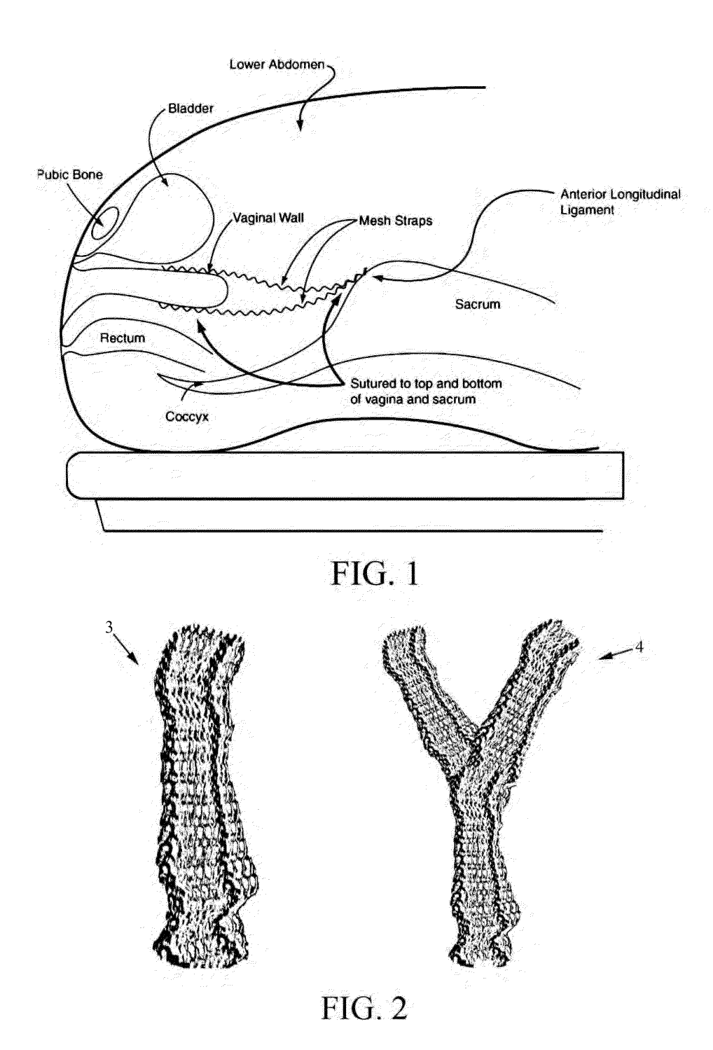

### DETAILED DESCRIPTION OF THE PREFERRED EMBODIMENTS

As described above, the present invention is a method and apparatus for introducing, positioning and anchoring a surgical mesh or implement freestanding at a surgical site on an anatomical tissue structure interiorly of the human body. The method and device can be used for all forms of surgical repair of the female condition vaginal prolapse, and is especially well suited for the minimally invasive laparoscopic approach where the surgical mesh is introduced into the female cavity through surgical ports (such a trocars). The present method and device, accurately positions, and reliably stabilizes the surgical mesh freestanding against vaginal tissue structure during the surgical procedure without handheld exterior support from surgical inserters or graspers. The invention may be used for a variety of different procedure types, such as for delivering surgical mesh, staples or other surgical tools or implements to a site during hernia, cholecystectomy (removal of the gall bladder) or other procedures. A preferred embodiment of the invention will herein be described in the context of a mesh delivery system for sacral colpopexy. The system employs a vaginal probe that is inserted vaginally, and a mesh stabilizer that is inserted laparoscopically through the abdominal wall into the lower abdominal cavity which then expands after insertion, and which magnetically docks through the vaginal tissue to the vaginal probe (interior in the vagina). The mesh stabilizer carries a payload of mesh, and can be left freestanding while docked for secure dispensing of mesh during the procedure, thereby overcoming alignment and positioning problems when placing and securing the surgical mesh on the vaginal tissue.

The probe tip of the vaginal probe has at least one and preferably an array of embedded magnetic member(s). The probe is inserted into the vagina and is positioned with the probe tip on one side of the vaginal apex, or other anatomical tissue structure for other procedures. A mesh stabilizer with its mesh payload is loaded (while in a compressed state) into a cartridge, the cartridge is loaded into a trocar or other port, and the surgical mesh assembly is then dispensed from the cartridge through the port into the abdomen using a conventional laparoscopic grasper/inserter or similar surgical tool. Once the mesh assembly passes through the port (inside the abdomen) the mesh assembly expands from its compressed state to an expanded state, and it is maneuvered inside the abdominal cavity by a grasper, needle driver or other instrument, into position at a desired surgical site against the tissue of the vaginal apex.

The mesh stabilizer likewise has at least one magnetic member attached and, once in position, the magnetic member is attracted to a magnetic member on the probe head which is positioned directly behind and inside the vaginal apex. This magnetically docks and stabilizes the mesh stabilizer at the surgical site on the anatomical tissue structure, e.g., vaginal apex. The payload of mesh attached to the mesh stabilizer can then be dispensed and adjusted on the vaginal tissue, the stabilizer keeping it tensioned and flat against the tissue of the vaginal apex during permanent suturing. When fixation is completed the mesh stabilizer is grasped and removed. This process of placing, holding and securing the surgical mesh to the vaginal apex is less time consuming, less prone to error than conventional surgical processes, and may facilitate transition to single-incision laparoscopic or robotic approaches to sacrocolpopexy which is less invasive thereby reducing morbidity for the patient as well as reducing overall hospitalized care.

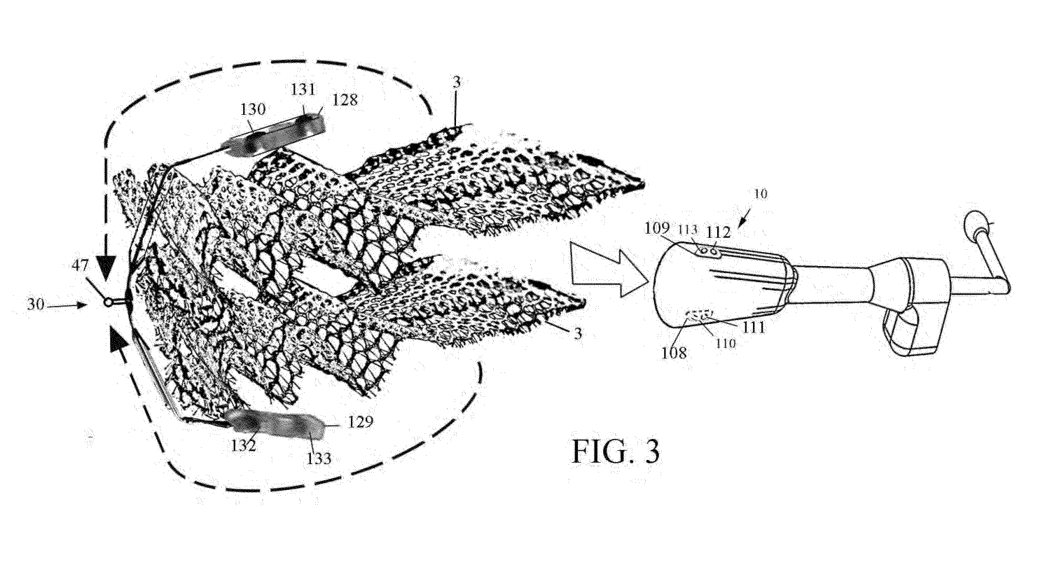

FIG. 3 illustrates the mesh stabilizer 30 containing two separate strips of mesh 3 (as per item 3 of FIG. 2), immediately before it docks and conforms to probe 10, thereby clamping the tissue of the vaginal apex (not shown) between mesh 3 and probe 10. The probe 10 may be any conventional vaginal probe modified as described below, and may be handheld, robotically-held, or adjustably held via a supporting framework to a support surface such as the operating table. In accordance with the stabilization aspect of the present invention, the probe 10 is equipped with at least one and preferably an array of magnetic members, for example, permanent magnets embedded in its tip. When a single magnetic member is used it may be recessed in the front center of the probe 10 head. However, the illustrated probe 10 embodiment includes a plurality (such as four) permanent magnetic disks 110-113 arranged in pairs on opposing sides of the probe 10 head as this indexes the mesh stabilizer 30 against inadvertent rotation. Specifically, a first pair of permanent magnetic disks 110, 111 is encased in a first plastic inset 108, and a second pair of permanent magnetic disks 112, 113 is encased in a second plastic inset 109. The insets 108, 109 are inlaid flush into recesses in the probe 10 head on opposing sides thereof as shown, and are bonded or otherwise adhered thereto. In use, the vaginally placed probe 10 is inserted into the vagina and essentially acts as a stabilizer for the vaginal tissue during dissection of the bladder and rectum away from the vagina and then during fixation of mesh to the vagina.

The mesh stabilizer 30 is a bent wire frame structure likewise having at least one and more preferably an array of corresponding magnetic members, for example, permanent magnets embedded in its tip. One skilled in the art should understand that if the magnetic member(s) on probe 10 are permanent magnets, the magnetic member(s) on mesh stabilizer 30 need may either be formed of attracted ferromagnetic material such as steel, or may be permanent magnets of opposing polarity, and vice versa. In the illustrated embodiment, opposing polarity permanent magnets are used on both. Thus, mesh stabilizer 30 has at least one permanent magnetic disk secured thereto, and preferably a plurality (four) permanent magnetic disks 130-133 in cooperative alignment with those of probe 10. As with the probe 10, in the illustrated embodiment, a first pair of permanent magnetic disks 130, 131 is encased in a first plastic shell 128 attached distally to a first side of the mesh stabilizer 30, and a second pair of permanent magnetic disks 132, 133 is encased in a second plastic shell 129 attached distally to the other side. The magnetic disks 130-133 may be molded inside shells 128, 129.

The magnetic disks of probe 10 and those of mesh stabilizer 30 align in pairs 113:130; 112:131; 110:132; 111:133, which allows for indexed relative linear and rotational positioning of the mesh stabilizer 30 against the vaginal apex. With the probe 10 (inserted vaginally) underlying the vaginal apex, the magnet(s) 130-133 of the mesh stabilizer 30 (inserted abdominally) are attracted to the magnets 110-113 of the probe 10 tip thereby anchoring the mesh stabilizer 30 to the vaginal apex inside the abdominal body cavity.

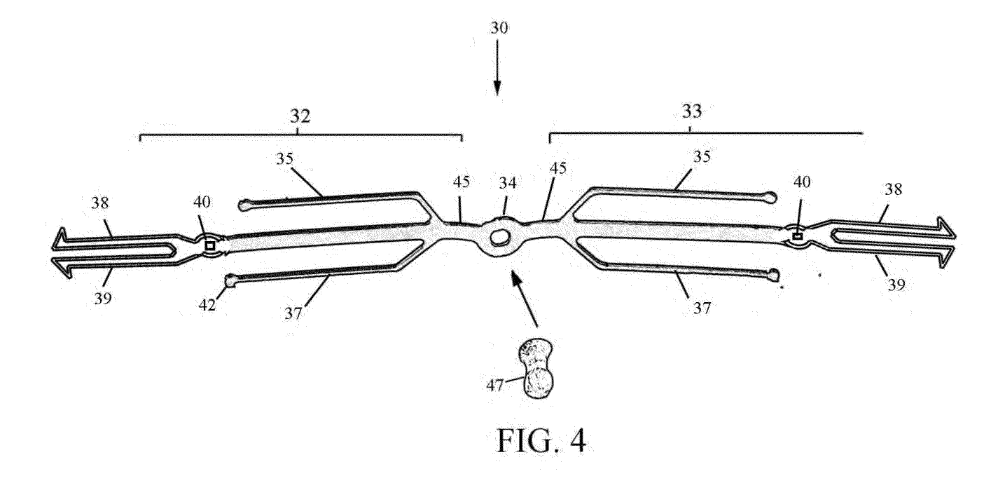

The mesh stabilizer 30 includes a wireframe body which may be formed by laser-cutting pseudoelastic material sheet stock into a flat wire frame structure (shown in FIG. 4 and described below), and then bending the flat structure into a three-dimensional shape (as depicted and described with regard to FIG. 3) conforming to the probe 10 head.

FIG. 4 is a front view of an exemplary mesh stabilizer 30 cut pattern prior to bending or attachment of permanent magnetic disks 130-133. Mesh stabilizer 30 may be laser cut from any resilient or pseudoeleastic sheet material in which deformation can be fully recovered upon unloading to the zero-stress state. Many metals exhibit pseudoelastic effects, but in the present context Ni—Ti based alloys (as well as other shape memory alloys) are preferred because of their material properties (super elastic) as well as their chemical and biological compatiblility with the human body. See, Castleman et al., “The Biocompatibility of Nitinol,” in Biocompatibility of Clinical Implant Materials, vol. 1, Williams DF (ed), CRC Press, p 129 (1981). The pseudoelastic alloy of the present invention preferably contains 55-56 percent Nickel and 44-45 percent Titanium, plus a remainder of one or more additional ternary alloying elements. A simple binary Ni—Ti of 56% Nickel and 44% Titanium is well suited and readily available from a variety of vendors including Norman Noble, a leading medical supplier. The mesh stabilizer 30 further comprises a first side 32 and a second side 33 joined together at a juncture 34, said first side and second side 32, 33 comprising diametrically-extending mesh stabilizing wire frameworks. The juncture 34 is for the attachment of a rearwardly-protruding ball-and-stem 47 for gripping, manipulating and releasing the mesh stabilizer 30, and for securing the ends of mesh 3, 4. The juncture 34 may also provide for attachment of a forwardly-facing magnetic member (not used in the current emvodiment), if so desired. In the preferred embodiment the mesh stabilizing wire frameworks on the first side and second side 32, 33 are mirror opposites. Each mesh stabilizing framework further comprises an elongate main stem 45, a pair of outwardly-protruding spring arms 35, 37 branching outward from along the main stems 45, and a distal pair of detent-prong fingers 38, 39 at the end of each stem 45 for slide-lock insertion into the plastic shells 12, 129 (see FIG. 3). The main stems 45 may optionally each be formed with a grasping feature 40, such as a loop (pictured), elbow or like feature for ease of grasping/manipulation in the jaws of a grasping instrument. The grasping feature 40 also allows the mesh stabilizer 30 to be gripped and manually open or closed. The protruding spring arms 35, 37 flare outward and angle parallel with main stems 45, and are preferably formed with distal tabs 40 for catching the weaves of the mesh 3, 4 to spread/tension it across the vaginal apex.

After initial laser-cutting as described above, the wireframe of mesh stabilizer 30 is permanently formed by bending into a three-dimensional shape with the first side 32 and second side 33 bent or arched at an angle in a V- or U-shape at juncture 34 in order to generally conform to the bulbous arc of the probe 10 head. This way, as seen in FIG. 3, the mesh stabilizer 30 attaches to the probe 10 head with the first side 32 overtop and second side 33 underneath. The four disc permanent magnets 130-133 couple to those of the probe 10 head as shown in FIG. 3, and the ball-and-stem 47 protrudes directly outward and rearward from the junction 34 to provide a means for grasping and ease of insertion/removal, and as well for temporarily securing the ends of mesh 3, 4.

Each mesh stabilizer 30 is adapted for carrying a payload of two strips of mesh 3, one for the upper vaginal apex and one for the lower. Alternatively, the mesh stabilizer 30 will accommodate a Y-shaped single piece of surgical mesh 4 (FIG. 2). The opposing main stems 45 both secure and dispense the onboard payload of mesh 3, 4 in accordance with an improved method of loading the payload and dispensing that is more efficient, accurate, and which reduces clutter and bunching.

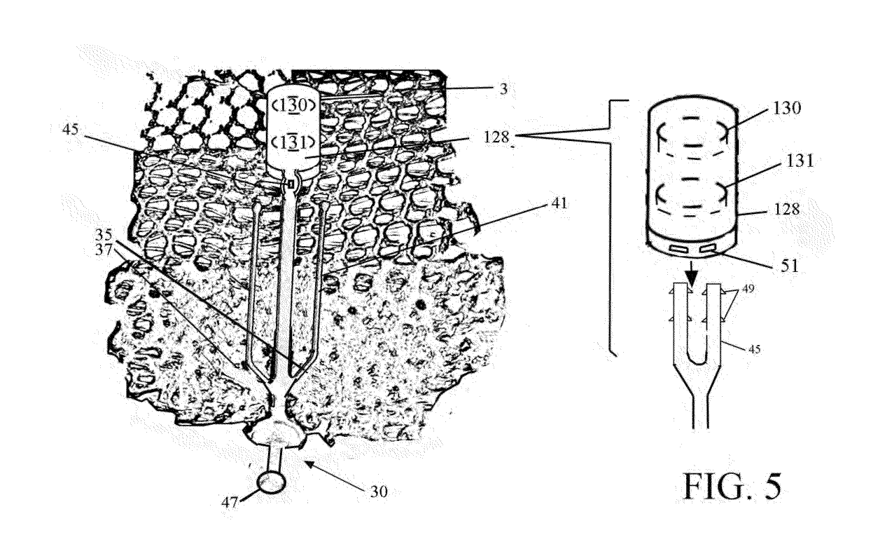

Specifically, payload loading comprises the following steps. If individual strips of mesh 3 are loaded into the mesh stabilizer 30 as seen in FIG. 5, one end of each strip 3 is placed at the junction 34 between the opposing main stems 45 and the other ends of the strips of mesh 3 are run outward around the shells 128, 129 (and encased magnetic members 130-133), and are folded back around the shells 128, 129 (as shown by dotted lines in FIG. 3) and temporarily pinned onto the protruding ball-and-stem 47. The diverging spring arms 35, 37 are highly resilient and penetrate the weaves of the mesh at two spaced points, the pads 42 retaining it in the loaded position. The spring arms 35, 37 are biased outward by the mesh 3, leaving a residual inward bias when the mesh 3 is fully loaded. This residual inward bias tends to clamp the mesh 3 under the spring arms 35, 37 (which are threaded through pores of the mesh 3) and prevents the mesh from falling off the arms 35, 37. The bias is sufficient to keep the mesh intact, flattening it against the vaginal apex, and yet it allows adjustment of the position of the mesh 3 relative to the mesh stabilizer 30 and vaginal apex while maintaining stabilization of the mesh 3 on the mesh stabilizer 30.

After docking of the loaded mesh stabilizer 30 to probe 10, the magnets 130-133 of mesh stabilizer 30 grip the corresponding magnets 110-113 embedded in the probe head and secure the mesh stabilizer 30 in place. The opposing resilient main stems 45 and spring arms 35, 37 of mesh stabilizer 30 conform to the vaginal tissue over the face of the probe 10 head both overtop and underneath, and at this point the inserter/grasper can be released from the ball-and-stem 47 and withdrawn. To then dispense the mesh payload, the ends of the strips of mesh 3 are unpinned and fed outward past the spring arms 35, 37 under the shells 128, 129 and encased magnetic members 130-133, the spring arms 35, 37 both spreading and flattening the mesh 3 as it is frictionally withdrawn under the magnetic members 130-133 and shells 129, 129. The outwardly-splayed spring arms 35, 37 release the weaves of the mesh 3 during extraction, but catch the mesh 3 if it tries to back up. The spring pressure of the main stems 45 and resilient spring arms 35, 37 in combination with the frictional withdrawal through (under) magnets 130-133 resists withdrawal, but the resistance can be easily overcome. Nevertheless, this resistance of the mesh strip 3 results in an incremental release, and the surgeon experiences a commensurate tactile feel as the mesh 3, 4 is released. The surgeon can more effectively index the exact length of mesh 3 that is extracted from mesh stabilizer 30 purely by feel. The free end of each mesh strip 3 or 4 may be extracted manually during the procedure to exactly the length needed for proper placement and fixation to the sacrospinous ligaments. This eliminates the propensity of loose portions of the mesh 3 to drape over and obscure the site of interest during fixation, and reduces the risk of the mesh 3 bunching or being sutured too tight.

The method of use is nearly the same for a single Y-shaped mesh 4 (as in FIG. 2) except that the main stem of the Y is folded accordion-style and seated into the trough of the bent and U-shaped mesh stabilizer 30 between the two diverging main stems 45 at junction 37. There is ample space within the trough of the (now-bent and UN-shaped) mesh stabilizer 30 to seat the accordion-folded end of Y-shaped mesh 4. The folding continues up the diverging strips of the Y-shaped mesh 4 exactly as described above and these two resulting stacks of folded mesh 4 are loaded onto the mesh stabilizer 30 as described. After the diverging strips of the Y-shaped mesh 4 have been secured to the vaginal tissue, the mesh stabilizer 30 can be removed leaving the main stem free for fixation to the sacrospinous ligaments.

FIG. 5 illustrates how the magnetic members 130, 131 are embedded in molded plastic shell 128 which is then attached to the main stem 45. The same configuration is used for magnetic members 132, 133 and shell 129. It is essential to minimize the risk of breakage of any components of the present invention, because if said components fall off they are likely to be left behind in the body cavity and cause post-surgical complications. If magnetic members 130, 131 were riveted or welded to main stems 45 there may be some risk of dislodgement, but retaining shells 128, 129 reduce any such risk. The retaining shells 128 are preferably thin elongate oval-shaped members with rounded ends and edges. The magnets 130, 131 may be molded inside plastic retaining shells 128, 129. Each retaining shell 128, 129 may be molded onto the distal end of main stem 45, or may be molded with one or more lengthwise channels 51 for insertion of main stems 45. In both cases main stem 45 may be formed with a plurality of flanking one-way teeth 49 that prevent extraction of main stem 45 from within the retaining shell 128, 129. In addition to securing magnets 130, 131, the retaining shells 12, 129 serve another purpose in that they diffuse the force of magnetic attraction across the entire face of retaining shells 128, 129. This reduces the point force of each magnet 130, 131 and makes it easier to slidably dispense mesh 3, 4 underneath.

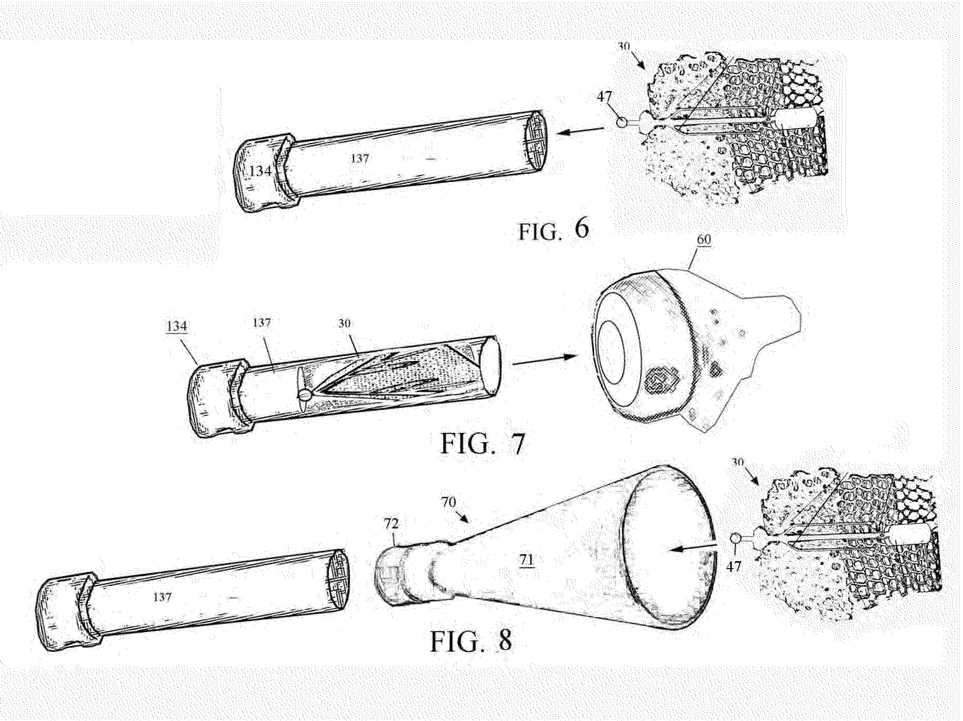

Given the above-described loading of a payload of mesh 3, 4 onto the mesh stabilizer 30, the stabilizer 30 itself is then loaded into a sterile packaging cartridge where it remains in a compact/compressed configuration for introduction by a standard grasper, needle driver, etc., through a laparoscopic port (such as a trocar) into the abdomen. There are several functional considerations which result in a variety of loading configurations. For example, the packaging cartridge must remain sterile, the mesh 3, 4 must remain in its uniform packed-payload configuration through the loading and deployment processes, and the stabilizer 30 and mesh 3, 4 must unfurl properly once ejected from the packaging cartridge. For example, FIG. 6 illustrates the packaging cartridge 137 which facilitates the placement of the mesh stabilizer 30 into a surgical port (such as a trocar) in preparation for introducing the mesh stabilizer 30 into a body cavity along with its payload of mesh 3, 4. The rudimentary way to get the stabilizer 30 and mesh 3, 4 inside the packaging cartridge 137 is to fold the spring arms 40 of mesh stabilizer 30 together (collapsed) with the preloaded mesh strips 3, 4 by squeezing the mesh stabilizer 30 with preloaded mesh 3, 4 laterally while simultaneously pulling or pushing the mesh stabilizer 30 and mesh 3, 4 into the packaging cartridge 137. In this manner, the mesh stabilizer 30 is fully and slidably preloaded into the packaging cartridge 137, cartridge 137 being capped with a collar 134, and is then sterilized and packaged for later use. Collar 134 is an elastomeric member that functions as a gas valve so it can work in conjunction with the trocar or similar surgical port. The collar 134 is essentially a cap with a central perforation that remains sealed around the protruding ball pin 47 on the mesh stabilizer 30 to maintain insufflation pressure when the tubular cartridge 137 is advanced into the laparoscopic trocar 60, yet still allowing passage of a laparoscopic grasper or needle driver through the collar 134 to advance the mesh stabilizer 30 into the abdomen. The collar 34 maintains insufflation pressure by sealing around the laparoscopic instrument, similar to other diaphragm valves as typically used in laparoscopic based procedures for allowing laproscopic access yet preventing the release of insufflation gases. The packaging cartridge 137 is a tubular member, preferably transparent, with rubber collar 134 mounted at one end. As seen in FIG. 7, the mesh stabilizer 30 loaded with mesh 3, 4 is pulled inside the packaging cartridge 137. The cartridge 137 is adapted for insertion through a standard trocar or port 60 to provide a passage into the body cavity. In this compact state, the main stems 45 and spring arms 35, 37 are constrained in a closed state for introduction through the surgical port (60) and into the abdomen. The preloaded packaging cartridge 137 may be placed in the surgical port 60 (FIG. 8). A surgeon can then easily introduce the mesh stabilizer 30 into the body cavity using standard laparoscopic tools. Since the mesh stabilizer 30 is compressed, the grasping tab 47 (FIG. 6.) remains fully accessible at the very center and is accessible by conventional laparoscopic tools to push the mesh stabilizer 30 through the port 60 into the abdomen (the cartridge 137 remains in the port 60). The natural expansion of the mesh stabilizer 30 deploys and unfurls the onboard mesh from a compressed configuration (unwrinkes the mesh).

In practice, the mesh stabilizer 30 may be packaged as a pre-loaded (or semi-preloaded) sub-assembly inside cartridge 137 as shown in FIG. 6, but is more preferably loaded in the cartridge by the surgeon just prior to the surgical procedure.

The inventors have found two recurring deployment issues and have developed solutions. The mesh 3, 4 must remain in its uniform packed-payload configuration through the loading and deployment processes, and the stabilizer 30 and mesh 3, 4 must unfurl properly once ejected from the packaging cartridge. Specifically, the inventors have found that if the mesh 3, 4 is loaded onto the mesh stabilizer 30, packed into the cartridge 137, and left unused for a prolonged period, the mesh 3, 4 has a tendency to retain its packed shape and may not adequately unfurl. Consequently, it is preferable to load the mesh 3, 4 onto the mesh stabilizer 30 and pack the assembly into the cartridge 137 just prior to the procedure. This reduces the shape memory characteristic of the mesh 3, 4 and assures adequate deployment, but requires packing by a surgeon or technician rather than the manufacturer. The inventors have also found that abruptly compressing the loaded mesh stabilizer 30 into the cartridge 137 can in some instances disrupt the mesh 3, 4, and the resulting disarray can make later dispensing of the mesh 3, 4 more difficult. FIG. 8 illustrates a packing funnel 70 which may be provided to simplify just-in-time packing of the mesh 3, 4 onto mesh stabilizer 30 and then into the cartridge 137. The packing funnel 70 is a hollow tube having a gradually tapered wall section 71 leading to an annular collar 72. The collar 72 is adapted for slidable insertion over the mouth of the cartridge 137. With packing funnel 70 mounted to cartridge 137, the packed mesh 3, 4 on mesh stabilizer 30 may be drawn into the cartridge 137 through the packing funnel 70, thereby gradually and automatically folding the packed mesh stabilizer 30 into its compact folded configuration within the cartridge 137. This is accomplished by inserting a conventional laparoscopic tool (e.g.—grasper or introducer) through the valved collar 134 and cartridge 137, grasping the protruding grasping tab 47, and pulling the mesh stabilizer 30 into the cartridge 137 through the packing funnel 70. The gradually tapered walls 71 avoid snagging and disruption of the mesh 3, 4. The packing funnel 70 is then removed with the mesh stabilizer 30 fully and slidably preloaded into the packaging cartridge 137, and cartridge 137 is ready for just-in-time use as described above. Packing funnel 70 greatly facilitates just-in-time packing and use of the mesh stabilizer 30 by attending physicians or nurses.

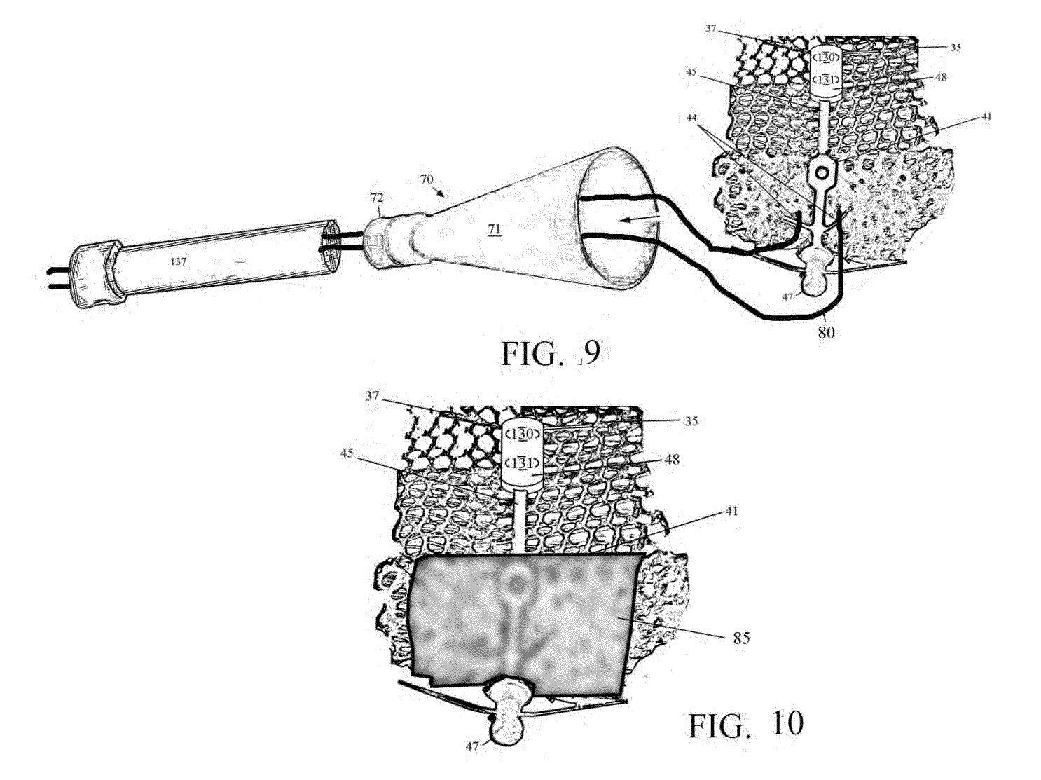

FIGS. 9-10 illustrate two additional (optional) features designed to facilitate loading and to maintain the accordion-folded configuration of mesh 3, 4 throughout loading and deployment.

FIG. 9 shows the use of a loading tether 80, which is a length of cord threaded through the valved collar 134 and cartridge 137, funnel 70, around the accordion-folded mesh payload 3, 4 on the mesh stabilizer 30, and back out as shown. Loading tether 80 is preferably wound around both folded mesh strips 3, 4 such that it serves as a lasso, binding the layers of mesh together. This way, rather than inserting a grasper or introducer through the valved collar 134, the surgeon need only grasp the loose ends of loading tether 80 and pull, drawing the mesh stabilizer 30 into the cartridge 137 through the packing funnel 70. Again, the gradually tapered walls 71 of funnel 70 avoid snagging and disruption of the mesh 3, 4 and maintain its folded configuration. Moreover, as the tether 80 is pulled it constricts both folded mesh strips 3, 4 and keeps the layers of mesh 3, 4 bound on their folded configuration throughout compaction in funnel 70 and loading of the mesh stabilizer 30 into cartridge 137. This further avoids disruption of the mesh 3, 4 and maintains a proper folded configuration. Once the mesh stabilizer 30 is packed the tether 80 can simply be removed by pulling one loose end.

FIG. 10 shows the use of a protective skirt 85, which is a panel of very thin but durable flexible low-friction sheet material such as Mylar™ or other plastic sheet, woven fabric, or the like. Protective skirt 85 is mounted centrally on the protruding tab 47 and forms a protective cover over the folded mesh 3, 4 maintaining a low friction surface between the mesh 3, 4 and interior of the funnel 70 and cartridge 137, protecting the folded mesh, and reducing the tendency of the mesh 3, 4 to catch or snag during loading. Any of the above-described loading procedures may be used inasmuch as the protective skirt 85 does not interfere, and once the cartridge 137 is loaded the protective skirt 85 may be removed or left in place through the procedure. One skilled in the art should understand that the above-described loading features including funnel 70, tether 80, and skirt 85 may be used alone or in combination, and in all such cases avoids disruption of the mesh 3, 4 and maintains a proper accordion-folded configuration during loading.

Once loaded, mesh stabilizer 30 may be introduced into the abdomen as described above where it is pushed out of the cartridge 137, expanding to the configuration shown in FIG. 3, and then magnetically docked to the vaginal probe 10 which precisely positions/anchors the mesh stabilizer 30 and its payload to the tissue of the vaginal apex inside the abdominal body cavity. The surgical tool used for introducing the mesh stabilizer is then removed. The docking engagement holds the mesh stabilizer 30 with mesh straps 3, 4 in place in the desired site with the muscular walls of the vagina lying between the vaginal probe and the mesh stabilizer. The surgeon is free to dispense mesh 3, 4 from the stabilizer 30 and/or adjust the position of the mesh relative to the vaginal apex and mesh stabilizer 30 while still maintaining stabilization of the mesh on the vaginal apex. This greatly facilitates fixation of the mesh to the anterior and posterior vaginal walls. After permanent fixation, driver surgical tool may be reinserted into the abdomen through the surgical port and used to retrieve the mesh stabilizer 30. The stability of the system results in a less complex and potentially less time consuming procedure, and may facilitate acceptance by many surgeons toward a minimally invasive approach (e.g.—single-incision laparoscopic or robotic approach) to sacrocolpopexy.

The present invention is suited for use with any surgical table, and both components 10, may be manually, mechanically or robotically manipulated. The vaginal probe 10 may be distally mounted on a flexible/locking stabilizing arm of a surgical table that thereby securely holds the probe 10 during the sacral colpopexy procedure (which indeed requires a stable probe during fixation of mesh to the vagina), or a manually supported probe.

When the mesh stabilizer 30 is deployed into the lower abdominal cavity, the opposing main stems 45 and spring arms 35, 37 and opposing foldable mesh 3, 4 unfurl to its open position (shown in FIG. 3). The probe 10 is inserted into a fixed opposing position within the vagina, and the mesh stabilizer 30 embraces and docks with the probe 10, collapsing around the top and bottom walls of the vaginal muscularis. When the mesh stabilizer 30 is fully docked with the probe 10 it sandwiches both the mesh and vaginal muscularis there between so that one strap of mesh 3, 4 sits opposed to the vaginal walls. This securely positions the mesh on the vaginal walls to which it will be secured, and adds some frictional resistance to withdrawal of the mesh through the closed-loop portion of the mesh stabilizer 30 stabilizing framework, thereby allowing the surgeon to adjust the position of the mesh relative to the vaginal apex and mesh stabilizer while still maintaining stabilization of the mesh on the vaginal muscularis.

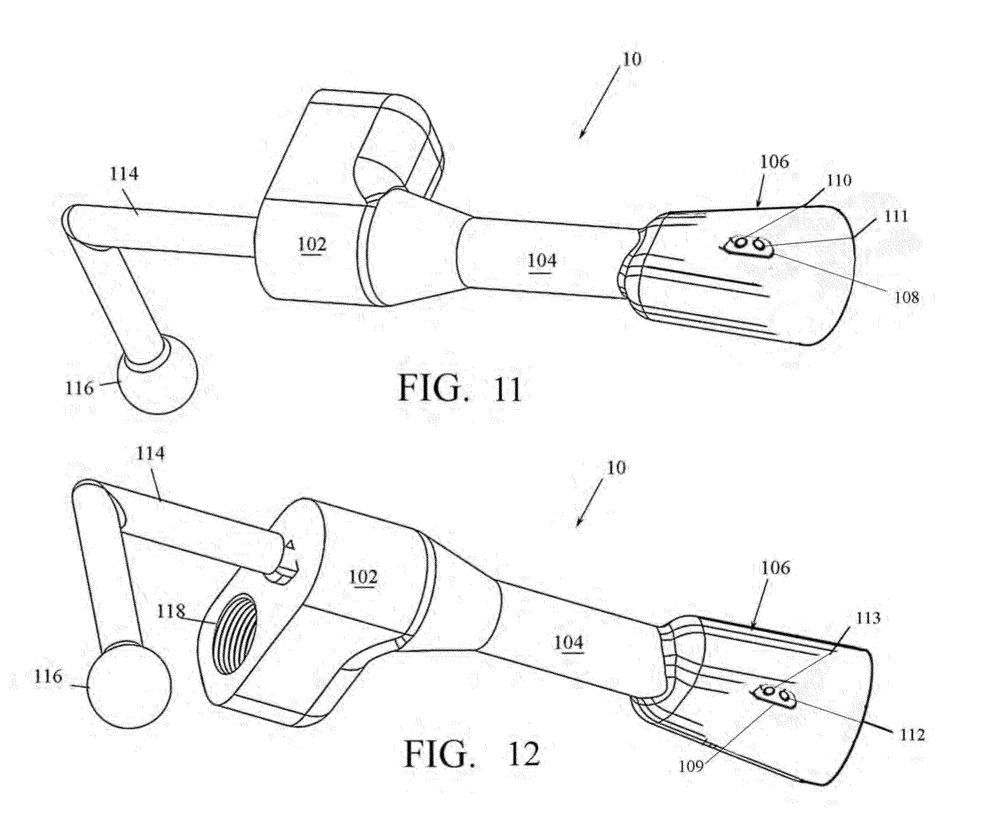

Virtually any vaginal probe may be modified for use with the present invention (including vaginal probes without magnets), but to improve stabilization of the mesh stabilizer 30 on the vaginal apex the magnetic attachment is recommended). FIGS. 11 and 12 are top and bottom side perspective views, respectively, of an exemplary vaginal probe 10. Probe 10 generally comprises a body 102 leading to a shaft 104 for insertion in the vagina, and a probe head 106 distal on the shaft 104. The probe head 106 is slightly flattened, with a generally oval horizontal and vertical cross-section flaring outward from the shaft 104, with rounded corners and edges so that it is more anatomically shaped to better conform the natural shape of the vagina than conventional vaginal probes. The probe head 106 may be tapered rearwardly of the tip to prevent inadvertent pop-off of the mesh stabilizer 30. Exemplary dimensions are 7 cm×5 cm×2.5 cm×4 cm, resulting in a 5 cm×2.5 cm probe end. The thicker tip can help prevent the spring arms 35, 37 of the stabilizer 30 from coming off (especially if the embodiment relies strictly on clamping). The shape of the probe head 106 may take on a variety of configurations as a matter of surgical discretion, and an exemplary set of probe head configurations specially suited for sacral colpopey are shown and described in co-pending U.S. Provisional Patent Application 61/636,171 filed 21 Apr. 2012.

In summary, after loading the mesh stabilizer 30 as described above, deployment generally includes six discrete steps: 1) opening; 2) coupling; 3) detachment; 4) extraction; 5) fixation, and 6) removal.

At 1) opening, the mesh stabilizer 30 is pushed into the abdomen with driver conventional laparoscopic tool, extending into abdomenal region. Once in the abdomen, the stabilizer 30 is exposed (freed from cartridge 137), and main stems 45/spring arms 35, 37 of the mesh stabilizer 30 open to a deployed U-or-V-shaped position as shown in FIG. 3.

At step 2) coupling, the open main stems 45 and spring arms 35, 37 are advanced over the vaginal apex and probe 10 head to begin the magnetic docking between the magnetic members of the mesh stabilizer 30 and the vaginal probe 10 head. The probe head 6 remains stationery.

At 3) detachment, the conventional laparoscopic tool that was used to introduce the mesh stabilizer 30 is removed leaving the stabilizer 30 attached to the vaginal apex and probe 10.

At 4) extraction, the mesh 3, 4 is manually extracted to the proper length and position for fixation in accordance with the method for extraction described herein.

At 5) fixation, the surgeon has an unobstructed view of the vaginal muscularis because the excess mesh straps are being contrained by the mesh stabilizer 30 and not hanging down and obscuring the surgeons vision, which facilitates the fixation of the mesh straps to the vaginal muscularis.

Upon completion of fixation, driver conventional laparoscopic tool can be reinserted and reattached to the stabilizer 30.

At removal 6) the mesh stabiler is removed from the abdomen back through the surgical port 60.

One skilled in the art should readily understand that there may be other mechanical mechanisms to achieve the requisite docking between the probe head 106 and mesh stabilizer 30, and the illustrated mechanisms are exemplary. In addition to the basic functionality described above, the probe 10 may be modified as desired to improve suitability to the task. For example, there may be one probe design for use with a flush vaginal vault, and one for use with a retained cervix. Alternatively, the vaginal probe 10 may be provided with a plurality of detachable tips for accomodating different vaginal configurations including the retained cervix. The vaginal vault probe may be equipped with a grasping mechanism at its tip to further stabilize the vagina and further minimize the risk of inadvertent pop-off of the mesh stabilizer 30 from the vaginal apex and the vaginal probe. The locking mechanism may be paired built-in grasping forceps, paired conical tips that prevent slippage without grasping, or paired suction channels to prevent slippage by creating a vacuum between the probe and the vaginal muscularis. A retained cervix vaginal probe must accommodate the cervix at its anterior tip. This may entail a shorter probe component that would sit within the endocervix to stabilize the cervix. Again, the probe 10 may contain some form of grasping component as described above to further stabilize the cervix and pull it flush against the probe.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

What is claimed is:

1. A method for loading a payload of surgical mesh onto a mesh stabilizer device for laparoscopic introduction into a human body cavity, comprising the steps of:
obtaining an elongate section of surgical mesh;
loading said section of mesh onto a pair of dispensing arms in said mesh stabilizer device such that said dispensing arms penetrate successive layers of said mesh through a weave of said mesh.

2. A surgical mesh stabilizer, comprising a wireframe elastic body formed with opposing mesh stabilizing frameworks joined together at a juncture, each of said mesh stabilizing frameworks including a pair of outwardly-protruding spring arms for tensioning and anchoring surgical mesh there beneath, and a pair of resilient dispensing arms diverging outward from said spring arm for insertion through an elongate section of surgical mesh folded accordion-style lengthwise such that said dispensing arms penetrate successive layers of said folded mesh through its pores.
